(19) United States
(12) Patent Application Publication
(10) Pub. No.: US 2017/0231827 A1
(43) Pub. Date: Aug. 17, 2017

(54) Hand or Foot Compression Wound Bandage System

(71) Applicant: Christina Goree, Arlington, TX (US)

(72) Inventor: Christina Goree, Arlington, TX (US)

(21) Appl. No.: 15/043,568

(22) Filed: Feb. 14, 2016

(51) Int. Cl. A61F 13/10; A61F 13/06; A61F 13/15; A61F 13/00

(52) U.S. Cl. CPC A61F 13/104; A61F 13/00017; A61F 13/00029; A61F 13/00042; A61F 13/00046; A61F 13/00068; A61F 13/064; A61F 13/15; A61F 2013/00238; A61F 2013/0028; A61F 2013/00285

(57) ABSTRACT

This invention relates to the field of a total covering breathable hand or foot compression bandage system that comprises a breathable hand compression wound bandage cover assembly; a breathable elastic fit compression sterile fabric hand or foot glove compression bandage insert which is inserted into the outer casing member of the hand compression wound bandage system; a plurality of sterile finger and toe tip absorbent pad bandages; adjustable cinching compression bandage straps that have a means to employ adjustable bandage straps to apply pressure to various potential user-selected wound locations; a wrist or ankle fastener which is integrated into the hand or foot compression wound bandage system at the wrist or ankle location to prevent the outflowing of blood or the entry of contaminates. Additional embodiments are a water proof model, have extra absorbency palm members that can apply extra wound pressure, or have pockets.

### FIELD OF THE INVENTION

This invention relates to the field of compression bandages and more particularly to sterile compression bandages that completely fit and enclose an injured hand or foot.

### BACKGROUND OF THE INVENTION

The present inventor recognized that first aid for hand and foot cuts and injuries suffer from many problems and/or shortcomings: First, self-adhesive bandage strips often impede dexterity and unravel when exposed to water. Second, when the hand injury occurs the blood flow impedes the person from immediately fulfilling the task at hand. These issues are particularly problematic for workers, such as chefs, barbers, firemen, emergency medical technicians, police, soldiers, and hairdressers, whose livelihoods depend on use of their hands.

This invention allows a sterile compression bandage to completely enclose the hand or foot while having additional capability to employ adjustable bandage straps to apply pressure to various potential would locations allowing the injured person to continue with immediate needed tasks. Moreover, the hand or foot compression bandage system has a wrist or ankle fastener at the end of the compression bandage system to prevent excess blood from flowing outside of the sterile hand or foot compression bandage system or contaminants from entering inside the hand or foot compression bandage system.

In order to address one or more of these needs and/or problems, the present inventor devised, several embodiments of the hand or foot compression wound bandage system.

One of the embodiments of the hand or foot compression wound bandage system invention comprises a hand compression wound bandage cover assembly with air flow perforation members; an elastic fit compression sterile fabric foot glove bandage insert with air flow perforation members which is inserted into the outer casing member of the hand compression wound bandage system; a plurality of sterile finger and toe tip absorbent pad bandages which are integrated into elastic fit sterile compression fabric hand or foot glove bandage inserts; adjustable cinching compression bandage straps that have a means to employ adjustable bandage straps to apply pressure to various potential wound locations on the hand compression wound bandage cover assembly which can be pre-attached at predetermined locations on the hand compression wound bandage cover assembly; a wrist or ankle fastener which is integrated into the hand or foot compression wound bandage system at the wrist or ankle location.

Another embodiment takes the form of a breathable latex or fabric glove with sterile lining and one more adjustable hook and loop fastener compression straps pre-attached or attachable to various points on the fingers or toes of the outside hand or foot glove assembly. The compression straps allow application of user-selected pressure at user-selected hand or finger/foot or toe locations to help control or stop bleeding.

In some embodiments, the glove includes an optional front or back pockets to hold anti-bacterial, additional compression straps, and/or analgesic ointments. In still other embodiments, the sterile absorbent lining is insertable and removable from the outer casing member of the hand or foot compression wound bandage system. In another embodiment the sterile glove inserts are a part of a whole disposable assembly of the hand compression wound bandage system.

In still other embodiments the hand or foot compression wound bandage system will take the form of a kit including two or more sterile liner gloves, an outer water-resistant glove, one or more adjustable hook and loop fastener straps, and one or more packages, for example three tear-open single-use antibacterial wipes, and an instruction sheet with first-aid instruction for burns and cuts. Other embodiments the hand or foot compression wound bandage system will take the form of separate supply kit for use with the hand or foot compression wound bandage system, with the kit including two or more replacement glove or finger liners and two or more single-use packages antibacterial wipes configured to fit a pocket of the glove.

In some embodiments, the fabric liner is formed of sterile gauze or a hydro entangled nonwoven web material Hydrofiber® dressings. In one embodiment, a translucent fabric gauze is used having a loose open weave structure in which the weft yarns are arranged in pairs and are crossed before and after each warp yarn. The breathable outer glove can be formed of a variety of materials, such as a microfiber, polyester fabric that moves perspiration away from the hand. Other embodiments use one or more of the following: acetate, nylon, acrylic, rayon, and spandex.

In some embodiments, the hand or foot compression wound bandage cover assembly are formed of a commercial synthetic rubber or other material distinct in form the base fabric of the outer glove.

Notable advantages of some embodiments are that bandage gloves without an extra strap allows water to enter, soaking any bandages so the invention has a hook and loop fastener to prevent this from occurring. This hand or foot compression wound bandage system first aid glove can be administered quickly and safely with or without assistance. Cross contamination is reduced with the use of the first aid glove.

Thus, the benefits of the invention surpass the current hand and foot bandage methods on the market.

Description of Prior Art

None of the bandage systems on the market that have been researched by the inventor had been found to have a sterile compression bandage that can completely enclose and bandage an injured hand or foot while having additional capability to employ adjustable bandage straps to apply pressure to various potential wound locations allowing the injured person to continue with immediate needed tasks.

For at least the foregoing reasons, there is a need for have sterile compression bandage that can completely enclose and bandage an injured hand or foot while having the additional capability to employ adjustable bandage straps to apply pressure to various user-selected potential wound locations allowing the injured person to continue with immediate needed tasks.

### SUMMARY

The present invention is directed to a total covering breathable hand or foot compression bandage system that comprises a breathable hand compression wound bandage cover assembly; a breathable elastic fit compression sterile fabric hand or foot glove compression bandage insert which is inserted into the outer casing member of the hand compression wound bandage system; a plurality of sterile finger and toe tip absorbent pad bandages; adjustable cinching compression bandage straps that have a means to employ adjustable bandage straps to apply pressure to various potential user-selected wound locations; a wrist or ankle fastener which is integrated into the hand or foot compression wound bandage system at the wrist or ankle location to prevent the outflowing of blood or the entry of contaminates.

Specific exemplary embodiments have been described. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of present teachings.

In some embodiments, the pocket opening is on the side or back. Some embodiments include a zipper closure to allow selective opening and closing of the pocket. Also some embodiments include analgesic and/or antiseptic packages, for example pre-moistened antiseptic and/or analgesic wipes, for use in cleaning a wound.

The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

Moreover in this document, relational terms, such as second, top and bottom, and the like may be used solely to distinguish one entity or action from another entity or action without necessarily requiring or implying any actual such relationship or order between such entities or actions. The terms “comprises,” “comprising,” “has”, “having,” “includes”, “including,” “contains”, “containing” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises a”, “has . . . a”, “includes . . . a”, “contains . . . a” does not, without more constraints, preclude the existence of additional identical elements in the process, method, article, or apparatus that comprises, has, includes, contains the element. The terms “a” and “an” are defined as one or more unless explicitly stated otherwise herein. The terms “substantially”, “essentially”, “approximately”, “about” or any other version thereof, are defined as being close to as understood by one of ordinary skill in the art, and in one non-limiting embodiment the term is defined to be within 10%, in another embodiment within 5%, in another embodiment within 1% and in another embodiment within 0.5%.

The term “coupled” as used herein is defined as connected, although not necessarily directly and not necessarily mechanically. A device or structure that is “configured” in a certain way is configured in at least that way, but may also be configured in ways that are not listed. Also, the term “exemplary” is used as an adjective herein to modify one or more nouns, such as embodiment, system, method, device, and is meant to indicate specifically that the noun is provided as a non-limiting example.

Factors and Aspects of the Invention

The first aspect of the invention of the hand or foot compression bandage system is that it can completely bandage and sterilely enclose an injured hand or foot, prevent blood loss and entry of outside contaminants while having additional capability to employ adjustable bandage straps to apply pressure to various user-selected potential wound locations allowing the injured person to continue with immediate needed tasks.

Second, the hand or foot compression bandage system allows for disposable elastic fit sterile compression hand or foot glove bandage inserts into the reusable hand or foot compression wound bandage cover assembly.

Third, the hand or foot compression bandage system has additional sterile finger and toe tip extra absorbent pad bandages integrated in the hand or foot compression bandage system.

Fourth, the hand or foot compression bandage system has additional sterile absorbency palm pad members allowing for extra wound compression and blood absorption integrated in the hand or foot compression bandage system.

For at least the foregoing reasons, there is a need for a hand or foot compression bandage system of this kind.

The foregoing and other objects, features, and advantages of the invention will become more apparent from the following detailed description, which proceeds with reference to the accompanying figures wherein the scale depicted is approximate.

### BRIEF DESCRIPTION OF THE DRAWING FIGURES

FIG. 1 is an angled back hand view of an embodiment of the present invention;

FIG. 2 is a palm left hand view of an embodiment of the present invention;

FIG. 3 is back left hand view of an embodiment of the present invention;

FIG. 4 is a left side palm hand view of an embodiment of the present invention;

FIG. 5 illustrates a close up of the sterile fingertip absorbent pad bandage;

FIG. 6 illustrates the elastic fit compression sterile fabric hand or foot glove bandage insert;

FIG. 7 illustrates a perspective view of the extra absorbency palm pad;

FIG. 8 is a perspective view of an embodiment of the foot compression wound bandage system.

### REFERENCE LISTING OF THE NUMBERED ELEMENTS

100 a hand or foot compression wound bandage system
110 foot compression wound bandage system
120 elastic fit compression sterile fabric hand or foot glove bandage insert
150 a hand or foot compression wound bandage cover assembly
170 sterile finger tip absorbent pad bandage
180 sterile toe tip absorbent pad bandage
190 adjustable cinching compression bandage straps
200 air flow perforation members
210 wrist fastener
220 ankle fastener
230 extra absorbency palm pad member
240 hook and loop fastener member of wrist or ankle fastener

### DEFINITIONS

In the following description, the term “insertable” as used herein, describes something which is capable of being inserted into something else.

In the following description, the term “sterile finger and toe tip absorbent pad bandages” as used herein, describes sterile finger and toe tip absorbent pad bandages which are more thick and liquid absorbent than the absorbent pads on regular standard box bandages and also provides extra compression wound force.

In the following description, the term “adjustable cinching compression bandage straps” as used herein, describes cinching straps that are capable of adjustable cinching strength that in turn provides adjustable compression wound force over the hand or glove bandage insert.

In the following description, the term “hand glove bandage insert” as used herein, describes an elastic fit compression sterile bandage which ergonomically fits and can be worn over the hand.

In the following description, the term “foot glove bandage insert” as used herein, describes an elastic fit compression sterile bandage which ergonomically fits and can be worn over the foot.

In the following description, the term “elastic fit compression sterile fabric hand or glove bandage insert” as used herein, describes an elastic fit compression hand or foot sterile bandage which is ergonomical and which is insertable into the hand or foot compression wound bandage cover assembly of the hand or foot compression wound bandage system.

In the following description, the term “a hand or foot compression wound bandage cover assembly” as used herein, describes the outer portion member of the hand or foot compression wound bandage system which covers and holds the elastic fit sterile fabric hand or glove bandage insert.

In the following description, the term “hand or foot compression wound bandage system” as used herein, describes either the hand compression wound bandage system or the foot compression wound bandage system which comprises a hand or foot compression wound bandage cover assembly; the said hand or foot compression wound bandage cover assembly having air flow perforation members; an elastic fit compression sterile fabric hand or foot glove bandage insert; the said elastic fit compression sterile fabric hand or foot glove bandage insert having air flow perforation members; the said elastic fit compression sterile fabric hand or foot glove bandage insert being capable of being inserted and removed from the said hand or foot compression wound bandage cover assembly; a plurality of sterile finger and toe tip absorbent pad bandages which are integrated into the said elastic fit compression sterile fabric hand or foot glove bandage insert at the finger or toe tip locations of the said elastic fit compression sterile fabric hand or foot glove bandage insert; a plurality of adjustable cinching compression bandage straps; the said adjustable cinching compression bandage straps being affixed to the said hand or foot compression wound bandage cover assembly at a plurality of locations; the said adjustable cinching compression bandage straps having a means that is commercially available today to apply adjustable compression wound force pressure to various potential wound locations on the said hand compression wound bandage cover assembly; a wrist or ankle fastener; the said wrist or ankle fastener which is integrated into the said hand or foot compression wound bandage cover assembly at the wrist or ankle location; the said wrist or ankle fastener having a cinching means that is commercially available today to prevent the outflow of blood and the inflow of outside contaminants from or into the said hand or foot compression wound bandage cover assembly.

The singular terms “a”, “an”, and “the” include plural referents unless the context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The term “comprises” means “includes.” All publications, patent applications, patents, and other references mentioned herein are incorporated by reference in their entirety for all purposes. In case of conflict, the present specification, including explanations of terms, will control. In addition, the materials, methods, and examples are illustrative only and not intended to be limiting.

### FIGURES

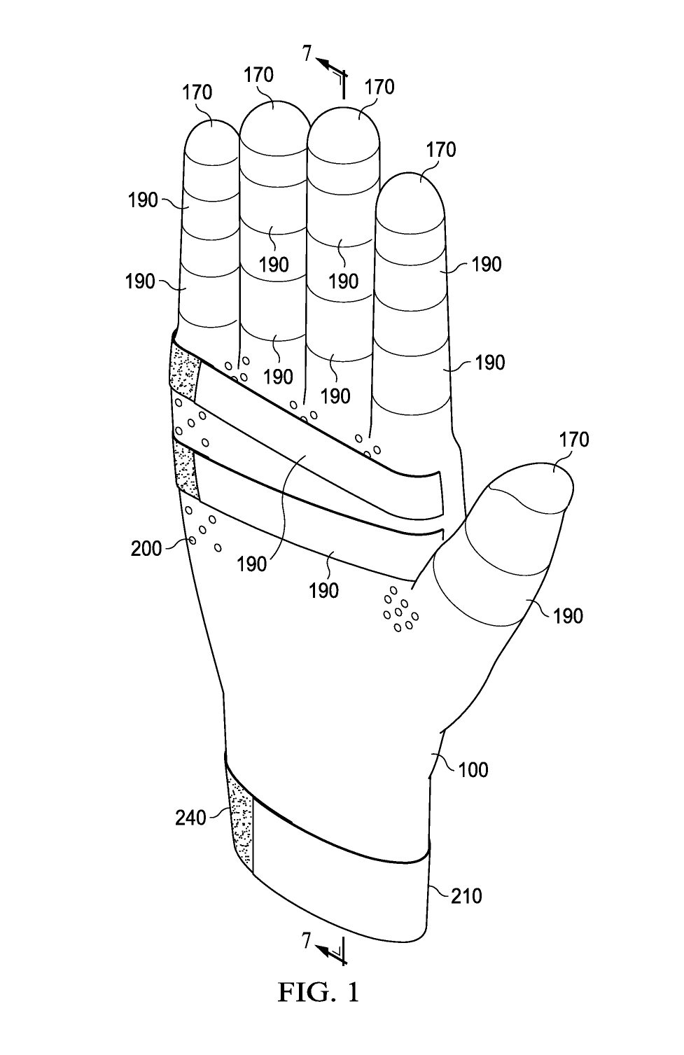

Referring generally to FIGS. 1-7, these figures illustrate embodiments according to the present invention for a hand or foot compression wound bandage system 100 and a method for bandaging hand or foot injuries using the hand or foot compression wound bandage system 100.

Referring to FIG. 1 illustrates an angled palm side facing view of a hand or foot compression wound bandage system 100. Moreover air flow perforation members 200 can be seen at varying placements on the hand or foot compression wound bandage system allowing air to flow between the hand and the air outside of the bandage Adjustable cinching compression bandage straps 190 can be seen on all of the fingers, thumb, and back of the hand.

The adjustable cinching compression bandage straps 190 can be tightened to the hold the desired wound pressure needed for the particular hand injury. Sterile fingertip absorbent pad bandages 170 illustrates a thick absorbent pad bandages which will allow for extra wound compression and blood absorption upon a fingertip or thumb tip injury. Further, FIG. 1 illustrates the wrist fastener 210 that the user can cinch up to prevent the outflow of blood and the entry of outside contaminants of the hand or foot compression wound bandage system 100. The wrist fastener 210 also shows the end of a hook and loop fastener member 240.

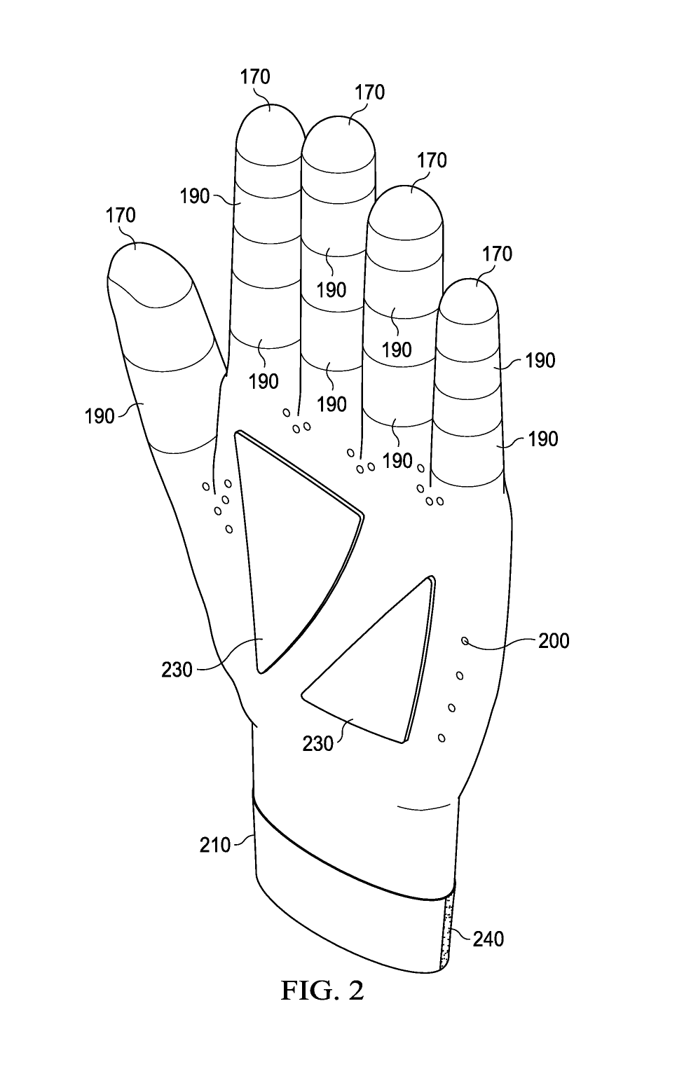

Referring to FIG. 2 illustrates a left hand angled palm view of an embodiment with the optional extra absorbency palm pad members 230. These extra absorbency palm pad members are designed to apply extra compression wound pressure to an inside palm injury.

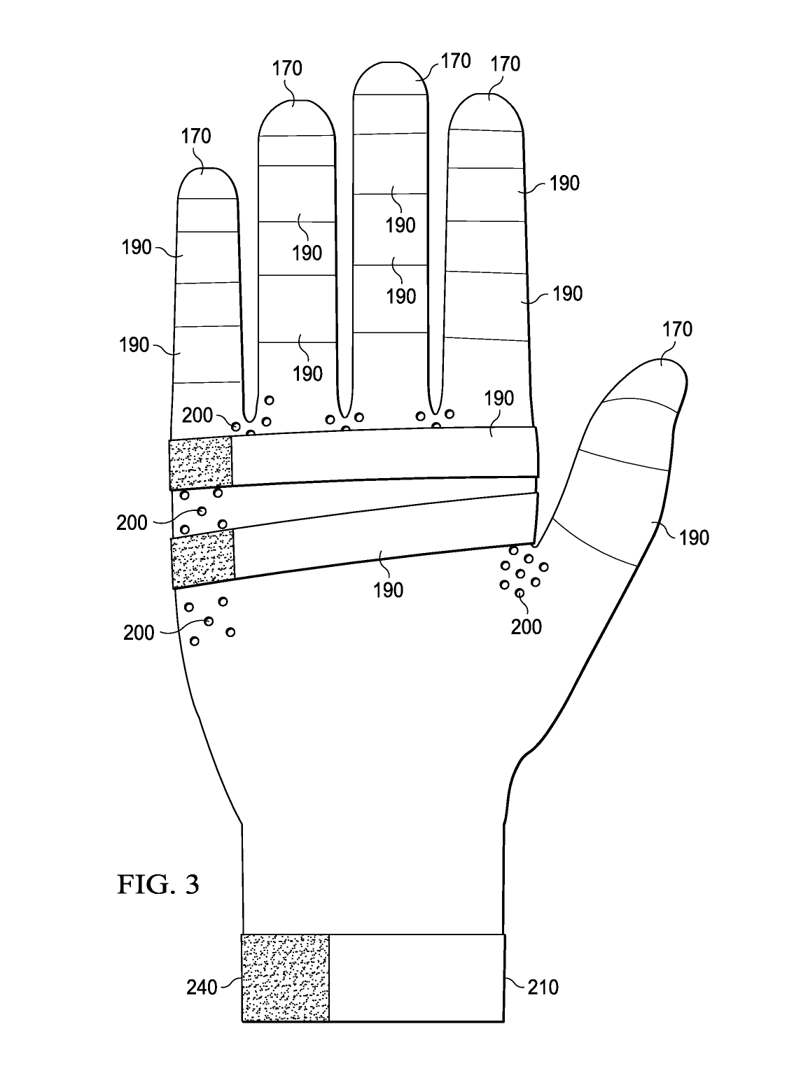

Referring to FIG. 3 illustrates a right side palm view of an embodiment of the invention.

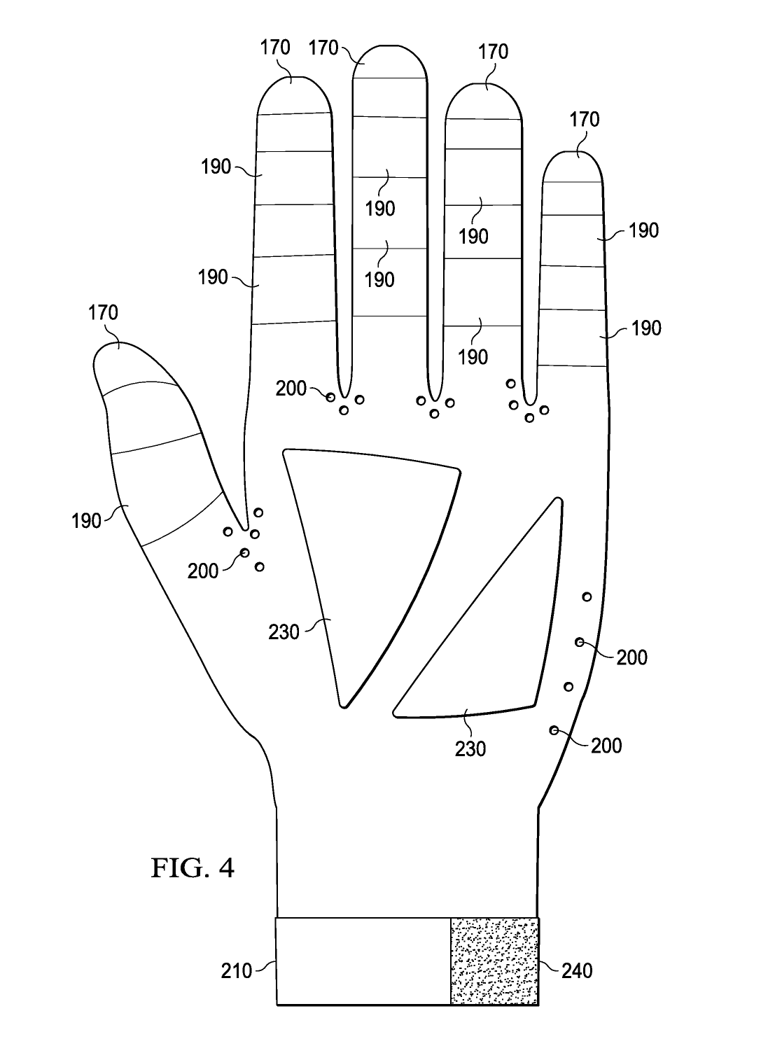

Referring to FIG. 4 illustrates a left side palm view of an embodiment of the invention.

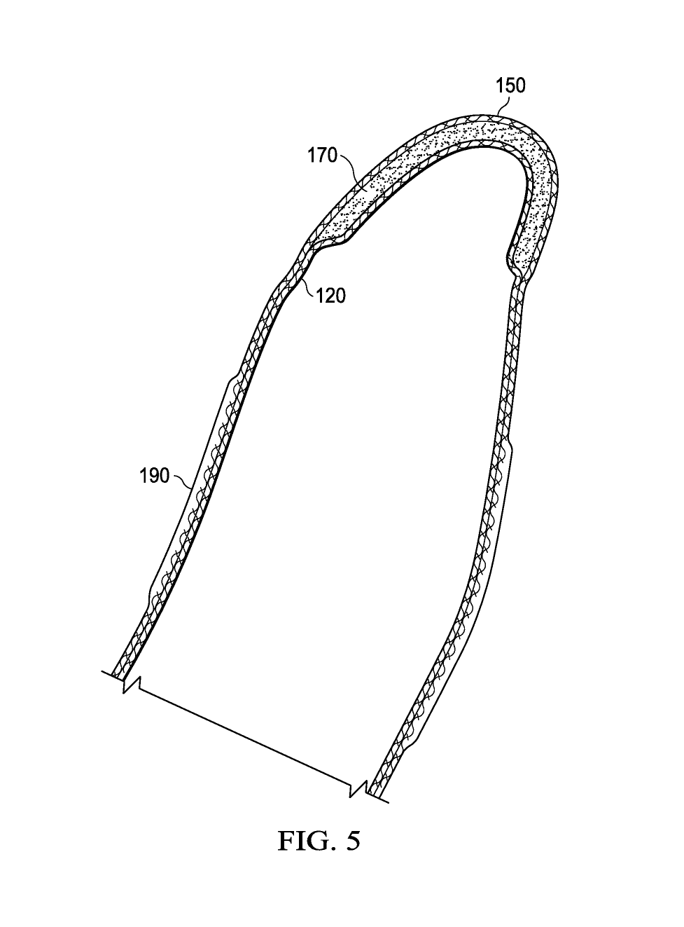

Referring to FIG. 5 a close up of the sterile fingertip absorbent pad bandage 170 on the outer side of the elastic fit compression sterile fabric hand or foot glove bandage insert 120. Moreover, an adjustable cinching compression bandage strap 190 is shown for reference. Further, the perspective of where the hand wound bandage cover assembly 150 is show in relation to the inserted elastic fit compression sterile fabric hand or foot glove bandage insert 120.

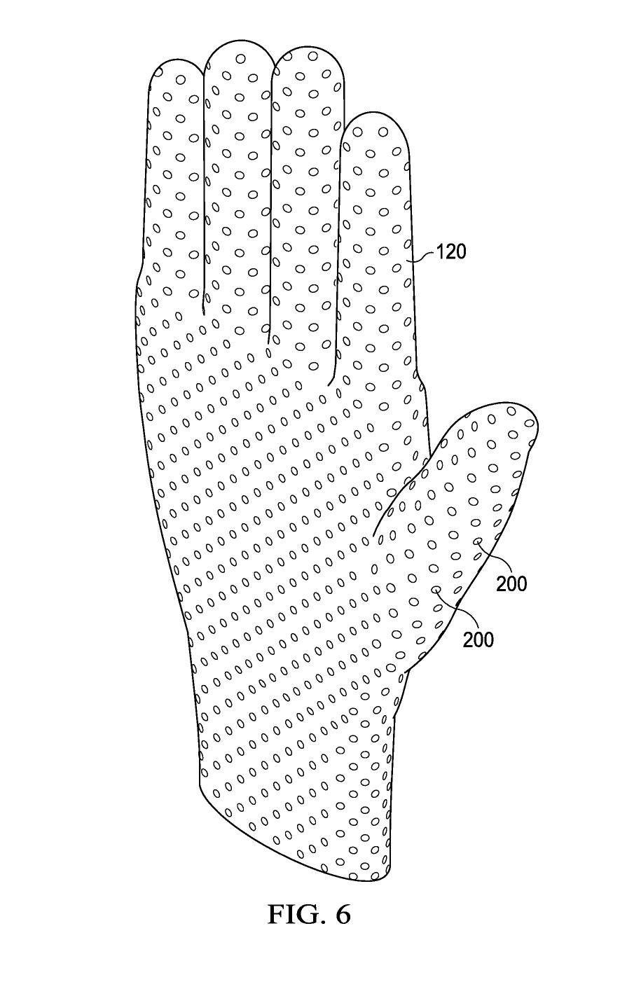

Referring to FIG. 6 illustrates an elastic fit compression sterile fabric hand or foot glove bandage insert 120 being used on a hand embodiment of the hand or foot 304 compression wound bandage system. Air flow perforation members 200 are also shown on the elastic fit compression sterile fabric hand or foot glove bandage insert 120.

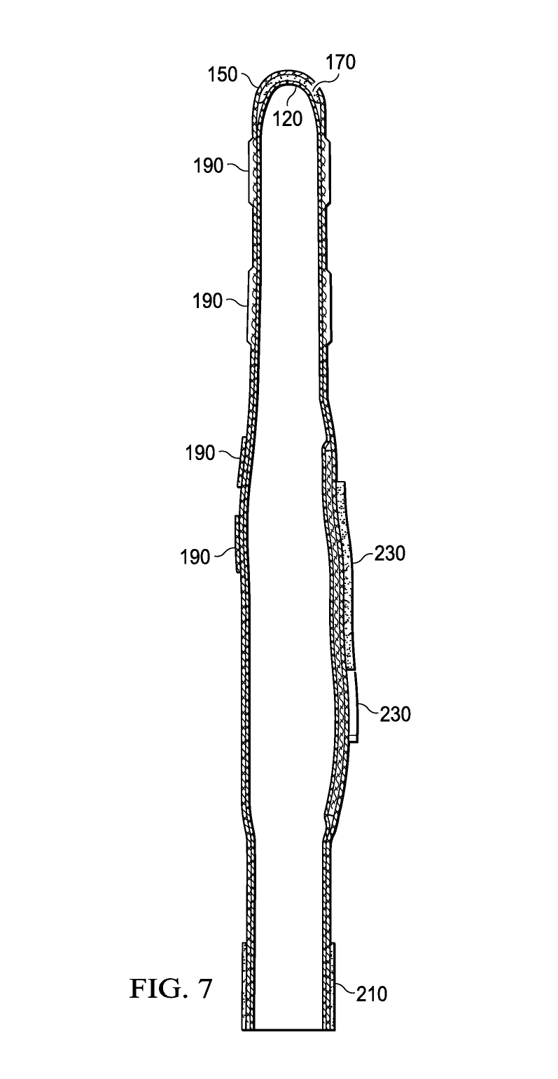

Referring to FIG. 7 illustrates the perspective that the extra absorbency palm pad member 230 being on the outside of an elastic fit compression sterile fabric hand or foot glove bandage insert 120 will provide extra compression wound force on the palm side of the hand. Again the sterile fingertip absorbent pad bandage 170 is also shown for additional illustration.

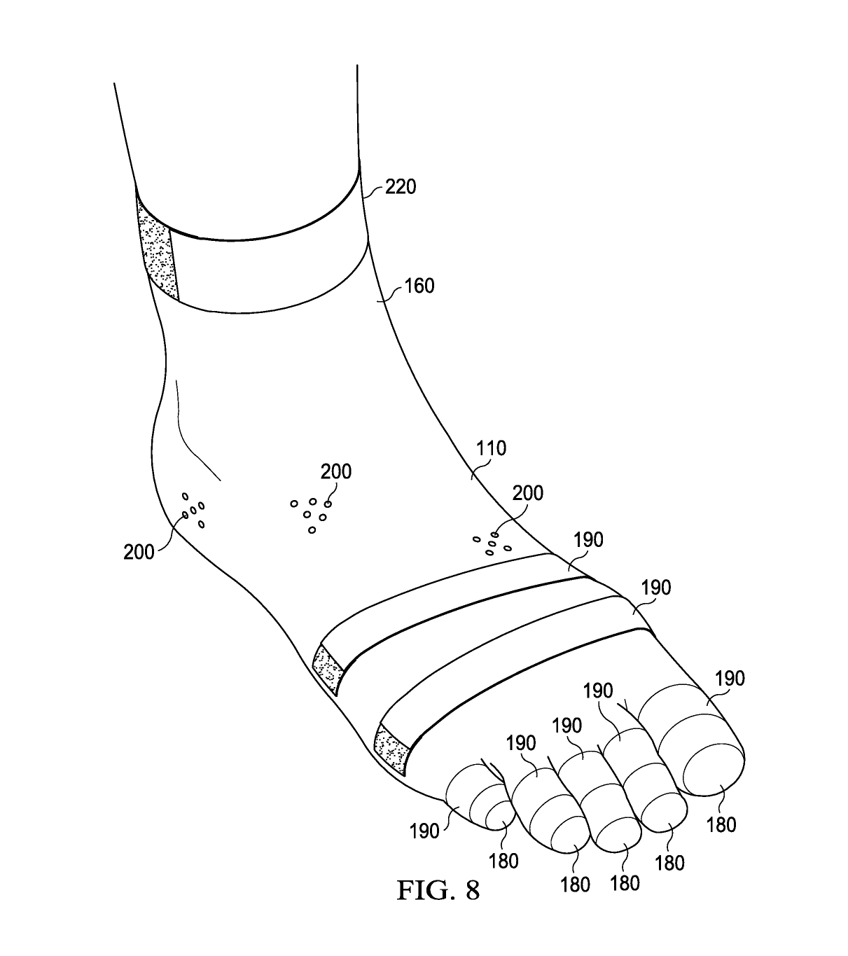

Referring to FIG. 8 illustrates an embodiment of a foot bandage system of the foot compression wound bandage system 110. Sterile toe tip absorbent pad bandages 180 are shown at all toe tips. Just as on the hand bandages embodiments, the foot embodiments of the hand or foot compression wound bandage system 110 have air flow perforation members 200 as well as adjustable cinching compression bandage straps 190.

It should be understood that the drawings and detailed description herein are to be regarded in an illustrative rather than a restrictive manner, and are not intended to he limiting to the particular forms and examples disclosed. Accordingly, it is intended that this disclosure encompass any further modifications, changes, rearrangements, substitutions, alternatives, design choices, and embodiments as would be appreciated by those of ordinary skill in the art having benefit of this disclosure, and falling within the scope of the following claims.

What is claimed is:

1. A hand or foot compression wound bandage system comprising:
a hand or foot compression wound bandage cover assembly; the said hand or foot compression wound bandage cover assembly having air flow perforation members; an elastic fit compression sterile fabric hand or foot glove bandage insert; the said elastic fit compression sterile fabric hand or foot glove bandage insert having air flow perforation members; the said elastic fit compression sterile fabric hand or foot glove bandage insert being capable of being inserted and removed from the said hand or foot compression wound bandage cover assembly; a plurality of sterile finger and toe tip absorbent pad bandages which are integrated into the said elastic fit compression sterile fabric hand or foot glove bandage insert at the finger or toe tip locations of the said elastic fit compression sterile fabric hand or foot glove bandage insert; a plurality of adjustable cinching compression bandage straps; the said adjustable cinching compression bandage straps being affixed to the said hand or foot compression wound bandage cover assembly at a plurality of locations; the said adjustable cinching compression bandage straps having a means that is commercially available today to apply adjustable compression wound force pressure to various potential wound locations on the said hand compression wound bandage cover assembly; a wrist or ankle fastener; the said wrist or ankle fastener which is integrated into the said hand or foot compression wound bandage cover assembly at the wrist or ankle location; the said wrist or ankle fastener having a cinching means that is commercially available today to prevent the outflow of blood and the inflow of outside contaminants from or into the said hand or foot compression wound bandage cover assembly.

2. A hand or foot compression wound bandage system according to claim 1 wherein the hand or foot compression wound bandage system is water resistant.

3. A hand or foot compression wound bandage system according to claim 1 wherein the hand or foot compression wound bandage system comprises a plurality of 3 1/16^{th} of an inch to ¼ of an inch thick extra absorbency hand or foot palm pad members.

4. A hand or foot compression wound bandage system according to claim 1 wherein the hand or foot compression wound bandage system contains pockets.

5. A hand or foot compression wound bandage system according to claim 1 wherein said hand or foot compression wound bandage cover assembly and the elastic fit compression sterile fabric hand or foot glove bandage insert are integrated as one unit.

6. A hand or foot compression wound bandage system according to claim 1 wherein the said hand or foot compression wound bandage cover assembly is made out of a commercial synthetic rubber such as neoprene.

7. A hand or foot compression wound bandage system according to claim 1 wherein the said elastic fit compression sterile fabric hand or foot glove bandage insert is made out of translucent fabric gauze having a loose open weave structure in which weft yarns are arranged in pairs and are crossed before and after each warp yarn.

8. A hand or foot compression wound bandage system according to claim 1 wherein the said hand or foot compression wound bandage cover assembly is made out of microfiber material.

9. A hand or foot compression wound bandage system according to claim 1 wherein the said hand or foot compression wound bandage cover assembly is made out of spandex.

10. A hand or foot compression wound bandage system according to claim 1 wherein the said hand or foot compression wound bandage cover assembly has at least one zipper enclosure mechanism.

11. A hand or foot compression wound bandage system according to claim 1 wherein the said elastic fit compression sterile fabric hand or foot glove bandage insert has at least one zipper enclosure mechanism.

12. A hand or foot compression wound bandage system according to claim 1 wherein the said hand or foot compression wound bandage cover assembly completely encloses and covers the injured hand or foot.

13. A hand or foot compression wound bandage system according to claim 1 wherein the said elastic fit compression sterile fabric hand or foot glove bandage completely encloses and covers the injured hand or foot.

14. A method of dressing a hand or foot wound using hand or foot compression wound bandage system comprising the steps of:
1) Providing a hand or foot compression wound bandage system comprising: a hand or foot compression wound bandage cover assembly; the said hand or foot compression wound bandage cover assembly having air flow perforation members; an elastic fit compression sterile fabric hand or foot glove bandage insert; the said elastic fit compression sterile fabric hand or foot glove bandage insert having air flow perforation members; the said elastic fit compression sterile fabric hand or foot glove bandage insert being capable of being inserted and removed from the said hand or foot compression wound bandage cover assembly; a plurality of sterile finger and toe tip absorbent pad bandages which are integrated into the said elastic fit compression sterile fabric hand or foot glove bandage insert at the finger or toe tip locations of the said elastic fit compression sterile fabric hand or foot glove bandage insert; a plurality of adjustable cinching compression bandage straps; the said adjustable cinching compression bandage straps being affixed to the said hand or foot compression wound bandage cover assembly at a plurality of locations; the said adjustable cinching compression bandage straps having a means that is commercially available today to apply adjustable compression wound force pressure to various potential wound locations on the said hand compression wound bandage cover assembly; a wrist or ankle fastener; the said wrist or ankle fastener which is integrated into the said hand or foot compression wound bandage cover assembly at the wrist or ankle location; the said wrist or ankle fastener having a cinching means that is commercially available today to prevent the outflow of blood and the inflow of outside contaminants from or into the said hand or foot compression wound bandage cover assembly;
2) Inserting a new fresh new said elastic fit compression sterile fabric hand or foot glove bandage insert inside the said hand or foot compression wound bandage system;
3) Downing the said hand or foot compression wound bandage system over the injured hand or foot;
4) The user will selected the location of the said cinching compression bandage straps that is over the cut or injury on the foot or hand and cinch that compression bandage strap to the desired wound compression pressure;
5) The user will than cinch the wrist or ankle fastener on the hand or foot compression wound bandage system.
